(19) United States
(12) Patent Application Publication
(10) Pub. No.: US 2018/0275135 A1
(43) Pub. Date: Sep. 27, 2018

(54) BIOMARKER PANEL TO IDENTIFY STEROID RESISTANCE IN CHILDHOOD IDIOPATHIC NEPHROTIC SYNDROME

(71) Applicant: Children's Hospital Medical Center, Cincinnati, OH (US)

(72) Inventors: Michael R. Bennett, Independence, KY (US); Prasad Devarajan, Cincinnati, OH (US)

(21) Appl. No.: 15/927,380

(22) Filed: Mar. 21, 2018

Related U.S. Application Data
(60) Provisional application No. 62/474,730, filed on Mar. 22, 2017.

(51) Int. Cl. G01N 33/68

(52) U.S. Cl. CPC G01N 33/6842; G01N 2800/52; G01N 2800/50; G01N 2800/60

(57) ABSTRACT

Disclosed are methods for the identification of an individual likely to have steroid resistant nephrotic syndrome (SRNS). The disclosed methods may include detection a plurality of proteins. The plurality of proteins may include, for example, Vitamin D Binding Protein (VDBP), Alpha-1 Acid Glycoprotein 2 (AGP-2), Fetuin A, prealbumin, and NGAL in a urine sample obtained from said individual. The methods may further include detection of Alpha-1 Acid Glycoprotein 1 (AGP-1), Alpha-1 B Glycoprotein (A1BG), Thyroxine binding globulin, hemopexin, and alpha-2 macroglobulin.

### CROSS-REFERENCE TO RELATED APPLICATIONS

This application claims priority to and benefit of U.S. Provisional Application 62/474,730, filed Mar. 22, 2017, the contents of which is incorporated in its entirety for all purposes.

### GOVERNMENT SUPPORT CLAUSE

This invention was made with government support under DK096418, awarded by the National Institute of Health. The government has certain rights in this invention

### BACKGROUND

Idiopathic nephrotic syndrome (NS) is the most common glomerular disease in children, occurring in 16 per 100,000 children. [1] Initial presentation of various NS subtypes are similar and include the presence of proteinuria, edema, hypoalbuminemia and hypercholesterolemia. Despite initial similarities, NS subtypes have markedly different disease courses and outcomes. Invasive biopsy remains the only method for positive diagnosis and the two most frequent histopathological findings are minimal change disease (MCD) and focal segmental glomerularsclerosis (FSGS). Prognosis depends on underlying pathophysiology and response to steroid treatment. Approximately 95% of children with MCD achieve remission following an 8-week course of prednisone (steroid sensitive nephrotic syndrome—SSNS) compared to 80% of patients with FSGS who fail to reach remission in response to steroids (steroid resistant nephrotic syndrome—SRNS). [2] FSGS is the most common acquired cause of end stage renal disease (ESRD) in children, and leads to further complications with roughly 30% recurrence post-transplant. [3, 4]

While kidney biopsies are effective for diagnosis in the adult population, they are not typically performed at presentation in children because response to therapy is a better indicator of long-term prognosis than histology in children, and FSGS is often underdiagnosed due to the smaller core size and focal nature of the disease. [2, 5] As a result, response to treatment is used as a one-size-fits-most diagnostic tool. The problem with this approach is that a population of children who are unlikely to respond to steroids (FSGS patients) are unnecessarily exposed to steroids and their potential side effects [6], and at the same time, postponing alternative treatments that may have a better chance of success. What are needed are non-invasive diagnostic tests that can predict which patients are more likely to respond to steroids to better inform caregivers to make the appropriate clinical decisions.

### BRIEF SUMMARY

Disclosed are methods for the identification of an individual likely to have steroid resistant nephrotic syndrome (SRNS). The disclosed methods may include detection a plurality of proteins. The plurality of proteins may include, for example, Vitamin D Binding Protein (VDBP), Alpha-1 Acid Glycoprotein 2 (AGP-2), Fetuin A, prealbumin, and NGAL in a urine sample obtained from said individual. The methods may further include detection of Alpha-1 Acid Glycoprotein 1 (AGP-1), Alpha-1 B Glycoprotein (A1BG), Thyroxine binding globulin, hemopexin, and alpha-2 macroglobulin.

### BRIEF DESCRIPTION OF THE DRAWINGS

Those of skill in the art will understand that the drawings, described below, are for illustrative purposes only. The drawings are not intended to limit the scope of the present teachings in any way.

FIG. 1. ROC curves using panels of 10 biomarkers (MLM-10) and 5 biomarkers (MLM-5) respectively

### DETAILED DESCRIPTION

### Definitions

Unless otherwise noted, terms are to be understood according to conventional usage by those of ordinary skill in the relevant art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

As used herein and in the appended claims, the singular forms “a,” “and,” and “the” include plural referents unless the context clearly dictates otherwise. Thus, for example, reference to “a method” includes a plurality of such methods and reference to “a dose” includes reference to one or more doses and equivalents thereof known to those skilled in the art, and so forth.

The term “about” or “approximately” means within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the art. Alternatively, “about” can mean a range of up to 20%, or up to 10%, or up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “alteration” (e.g., an increase or decrease) in the level of a biomarker (for example in a sample obtained from a subject) relative to the level of a corresponding protein in a control sample, is indicative of the status of the subject as steroid resistant or steroid sensitive.

The terms “individual,” “host,” “subject,” and “patient” are used interchangeably to refer to an animal that is the object of treatment, observation and/or experiment. Generally, the term refers to a human patient, but the methods and compositions may be equally applicable to non-human subjects such as other mammals. In some embodiments, the terms refer to humans. In further embodiments, the terms may refer to children.

The term “biomarker” as used herein refers to a peptide, protein, or nucleic acid in a subject or in a biological sample obtained from a subject, whose presence and/or level is indicative of a biological process, pathogenic process, or pharmacologic response to therapeutic intervention. In one aspect, detection of the biomarker may be used to predict therapeutic outcome or likelihood of a subject being responsive to a particular treatment.

Idiopathic nephrotic syndrome (NS) is the most common glomerular disorder of childhood. Prognosis correlates with steroid responsiveness, from sensitive (SSNS) to resistant (SRNS). SRNS is the most common acquired cause of end stage renal disease (ESRD) in children. Non-invasive biomarkers that could predict steroid resistance would help patients avoid unnecessary exposure to high dose corticosteroids and help to tailor treatments with alternative drugs that are more likely to be beneficial. Here, Applicant has discovered and performed an initial validation of a candidate biomarker panel that differentiates steroid resistance in children with nephrotic syndrome and correlates with poor renal function.

In one aspect, a method for identifying an individual likely to have steroid resistant nephrotic syndrome (SRNS) is disclosed. The method may comprise the step of detecting a plurality of proteins. The plurality of proteins may comprise one or more of Vitamin D Binding Protein (VDBP), Alpha-1 Acid Glycoprotein 2 (AGP-2), Fetuin A, prealbumin, and NGAL. The plurality of proteins may be detected in a urine sample obtained from an individual likely to have steroid resistant nephrotic syndrome.

In one aspect, the method may comprise the step of quantifying a plurality of proteins. The method may further comprise the step of identifying the presence of an alteration in the level of each protein in the plurality of proteins. An alteration may be indicative of said individual being steroid sensitive or steroid resistant. In one aspect, the method may comprise the step of quantifying one or more proteins in the plurality of proteins and applying an algorithm such as that set forth in Column 2 of Table 7 herein.

In one aspect, the detection step may be carried out using a 4-plex isotope tagging method (iTRAQ).

In one aspect, the method may comprise the step of detecting Alpha-1 Acid Glycoprotein 1 (AGP-1), Alpha-1 B Glycoprotein (A1BG), Thyroxine binding globulin, hemopexin, and alpha-2 macroglobulin in said sample, and may further comprise the step of quantifying said proteins and applying the algorithm set forth in Column 1 of Table 7. The individual may then be characterized as either steroid resistant or steroid sensitive. In one aspect, application of the algorithm of the method may be computer-implemented.

In one aspect, the individual may be diagnosed with nephrotic syndrome prior to conducting the aforementioned steps.

In one aspect, a method of identifying a steroid resistant individual diagnosed with nephrotic syndrome is disclosed. The method may comprise the steps of contacting a urine sample from said individual with a composition comprising a plurality of detecting agents, wherein said plurality of detecting agents are capable of detecting a protein selected from Vitamin D Binding Protein (VDBP), Alpha-1 Acid Glycoprotein 2 (AGP-2), Fetuin A, prealbumin, and NGAL or a combination thereof. In one aspect, the composition may further comprise a plurality of detecting agents capable of detecting a protein selected from Alpha-1 Acid Glycoprotein 1 (AGP-1), Alpha-1 B Glycoprotein (A1BG), Thyroxine binding globulin, hemopexin, and alpha-2 macroglobulin. The detecting agent may be, for example, an antibody.

In one aspect, a kit for classifying a subject diagnosed with nephrotic syndrome as steroid sensitive or steroid sensitive is disclosed. The kit may comprise a set of detection agents consisting of detection agents capable of detecting the expression products of 5 different biomarkers in a test sample, or 10 different biomarkers in a test sample, wherein said 5 different biomarkers may be Vitamin D Binding Protein (VDBP), Alpha-1 Acid Glycoprotein 2 (AGP-2), Fetuin A, prealbumin, and NGAL and wherein said 10 different biomarkers may comprise Vitamin D Binding Protein (VDBP), Alpha-1 Acid Glycoprotein 2 (AGP-2), Fetuin A, prealbumin, NGAL, Alpha-1 Acid Glycoprotein 1 (AGP-1), Alpha-1 B Glycoprotein (A1BG), Thyroxine binding globulin, hemopexin, and alpha-2 macroglobulin. The kit may further comprise a computer product for calculating a value for a subject according to Table 7, wherein said value is predictive of said classification.

### Examples

The following non-limiting examples are provided to further illustrate embodiments of the invention disclosed herein. It should be appreciated by those of skill in the art that the techniques disclosed in the examples that follow represent approaches that have been found to function well in the practice of the invention, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the invention.

Urine and clinical data were collected from 50 patients, aged 2-19 that were diagnosed with idiopathic nephrotic syndrome at Cincinnati Children's Hospital Medical Center. Isobaric tags for relative quantitation (iTRAQ) was used to discover 13 proteins that were differentially expressed in SSNS vs SRNS in a small 5×5 discovery cohort. Suitable assays were found for 9 of the 13 markers identified by iTRAQ and were used in a 25 SRNS×25 SSNS validation cohort. Vitamin D Binding Protein (VDBP), Alpha-1 Acid Glycoprotein 1 (AGP-1), Alpha-1 Acid Glycoprotein 2 (AGP-2), Alpha-1 B Glycoprotein (A1BG), Fetuin A, prealbumin, Thyroxine binding globulin and hemopexin, alpha-2 macroglobulin were measured and combined with urine NGAL which had been previously shown to distinguish patients with SRNS. Urinary Vitamin D-binding protein, Prealbumin, NGAL, Fetuin A, and AGP2 were found to be significantly elevated in SRNS using univariate analysis, with AUCs ranging from 0.65-0.81. Multivariate analysis revealed a panel of all 10 markers that yielded an AUC of 0.92 for identification of SRNS. A subset of 5 markers (including VDBP, NGAL, fetuin A, prealbumin, and AGP2) showed significant associations with SRNS and yielded an AUC of 0.85.

Applicant enrolled pediatric patients with idiopathic nephrotic syndrome and compared the urine proteome of patients with SSNS to those with SRNS. iTRAQ labeling techniques were used for relative quantitation and identification of differentially-expressed proteins. These methods, which originated from the isotope-codes affinity tag (ICAT) approach reported by Gygi et al. [7], have the added advantages of using labeling chemistry targeted at primary amines (rather than sulfhydryl groups) and the ability to simultaneously measure relative quantities of proteins under multiple conditions. [8] Differentially expressed proteins were validated using clinically available tools such as ELISA and clinical immunonephelometry.

Patients and Study Design

Under an IRB-approved protocol, informed consent was recorded from all participants and/or their legal guardians. Exclusion criteria included: history of gross hematuria, active or recurrent urinary tract infection or nephrotic syndrome secondary to systemic disease. Urine and clinical data were collected from 50 patients, aged 2-19 that were diagnosed with idiopathic nephrotic syndrome at Cincinnati Children's Hospital Medical Center. The samples were collected over a period of 24 months. The study included 20 patients with SRNS (19 of whom had biopsy proven FSGS), and 30 patients with SSNS. Urine was collected as part of a standard clinical visit, centrifuged at 5000 g for 5 minutes, aliquoted, and stored at −80° C. No more than 2 freeze-thaw cycles were used per sample. Demographic and clinical data, including urinalysis, steroid-response history, most recent serum creatinine, and current remission/relapse status were recorded at the time of patient enrollment. Estimated glomerular filtration rate (eGFR) was calculated from serum creatinine using the new Schwartz Formula [9] and classified to chronic kidney disease (CKD) stage. [10] SSNS was defined as the ability to reach remission within 8 weeks after initial diagnosis in response to steroid treatment, as evidenced by normalization of protein urine reading to a negative reading on a urine dipstick. SRNS was defined as a failure to respond to standard steroid treatment (2 mg/kg/day) for at least 8 weeks.

Quantitative profiling of urine proteins using isobaric protein labeling and tandem mass spectrometry.

Urine samples from two subject groups (5 each from SRNS relapse and SSNS relapse) were prepared for quantitative protein profiling using the iTRAQ method [8] by following the vendor instructions (Sciex). The sample preparation protocol prior to iTRAQ tagging varied from the original vendor protocol, thus the workflow is summarized here with details of each step provided below. The general sample preparation and analysis workflow included concentration and buffer exchange of each urine sample followed by preparative separation of the proteins on a mini SDS-PAGE gel, in gel trypsin digestion and recovery of the peptides, iTRAQ tagging of duplicate SSNS and SRNS samples with the iTRAQ 4-plex reagents (114, 115, 116, 117 reporters), combining the peptide from the 4 samples in equal portions, then subjecting peptides to nanoLC-MSMS, followed by protein identification and quantitation of the collective data set using the ProteinPilot (PP), ProteinPilot Descriptive Statistics Template (PDST) and Protein Alignment software algorithms (AB Sciex). Additional details of each step in the process are provided below.

Gel electrophoresis and isolation of peptides. Protein from SRNS and SSNS urine samples were concentrated, buffer exchanged (2×) with Invitrogen 1× Laemmli buffer using 3 kDa amicon concentrator cartridge (UFC500396). The protein concentration for each sample was determined using the non-interfering (Ni) protein assay reagents from G-Biosciences (Maryland Heights, Mo.). 50 ug each from the 5 SRNS and 5 SSNS (10 samples total) were loaded onto separated lanes of a 1D, 4-12% Bis-Tris minigel, then electrophoresed for 15 min which was just long enough for the proteins to enter into the gel. The gel region containing the proteins (about 1.5 cm×2.5 cm) was cut from the gel and subjected to in gel trypsin digestion and subsequent recovery of peptides as described previously. [11]

iTRAQ labeling. The isolated peptides from the 10 urine samples (5 SRNS and 5 SSNS) were each divided in half such that technical replicates were available for each sample. 5 pairwise comparative groups (A-E) were tagged using the 4-plex iTRAQ reagents as described previously. [8] The 114 and 115 reporter tags were used for the technical replicates of SRNS samples while the 116 and 117 reporter tags were used for the technical replicates of the SSNS samples. After labeling the samples were mixed together in equal quantities for subsequent separation, identification and quantitative analysis.

Nano liquid chromatography coupled electrospray tandem mass spectrometry (nLC-ESI-MS/MS). nLC-ESI-MS/MS analyses were performed on a TripleTOF 5600+(Sciex, Toronto, ON, Canada) attached to an Eksigent (Dublin, Calif.) nanoLC.ultra nanoflow system. 2.5 ug of total protein from each 4-plex mixture was loaded (via an Eksigent nanoLC.as-2 autosampler) onto an IntegraFrit Trap Column (outer diameter of 360 μm, inner diameter of 100, and 25 μm packed bed) from New Objective, Inc. (Woburn, Mass.) at 2 μl/min in formic acid/H2O 0.1/99.9 (v/v) for 15 min to desalt and concentrate the samples. For the chromatographic separation of peptides, the trap-column was switched to align with the analytical column, Acclaim PepMap100 (inner diameter of 75 μm, length of 15 cm, C18 particle sizes of 3 μm and pore sizes of 100 Å) from Dionex-Thermo Fisher Scientific (Sunnyvale, Calif.). The peptides were eluted using a variable mobile phase (MP) gradient from 95% phase A (Formic acid/H2O 0.1/99.9, v/v) to 40% phase B (Formic Acid/Acetonitrile 0.1/99.9, v/v) for 70 min, from 40% phase B to 85% phase B for 5 mins and then keeping the same mobile phase composition for 5 more minutes at 300 nL/min. The nLC effluent was ionized and sprayed into the mass spectrometer using NANOSpray® III Source (AB Sciex, Toronto, On, Canada). Ion source gas 1 (GS1), ion source gas 2 (GS2) and curtain gas (CUR) were respectively kept at 7, 0 and 25 vendor specified arbitrary units. Interface heater temperature and ion spray voltage was kept at 150 C, and at 2.3 kV respectively. Mass spectrometer method was operated in positive ion mode set to go through 4156 cycles for 90 minutes, where each cycle performing one TOF-MS scan type (0.25 sec accumulation time, in a 400 to 1600 m/z window) followed by twenty information dependent acquisition (IDA)-mode MS/MS-scans on the most intense candidate ions having a minimum 150 counts. Each product ion scan was operated under vender specified high-sensitivity mode with an accumulation time of 0.05 secs and a mass tolerance of 50 mDa. Former MS/MS-analyzed candidate ions were excluded for 10 secs after its first occurrence, and data were recorded using Analyst®-TF (v.1.6) software.

Data Analyses of Quantitative Protein Profiling

Individual and merged search from the nLC-MS/MS analyses were accomplished using ProteinPilot software (version 4.5, revision 1656) that utilizes Paragon algorithm, against a SwissProt database of human protein sequences. A vendor sample type including all biological modification was selected for the search parameter as variable modification while methylthiocysteine was used as a fixed modification. The output files for the ProteinPilot database search (*.group file) contain the peptide identification tables, protein identification tables and relative quantitation data from the iTRAQ reporter ions from each peptide all of which can be exported as Excel spreadsheets for further statistical analysis using the ProteinPilot Descriptive Statistics Template (PDST, ver 3.005pB).[12] The PDST is a mathematical Excel template that processes the relative quantitation data among the sample sets and provides statistical probabilities related to the confidence of the protein identification in relationship to an inverse (decoy) protein database, and provide p values regarding the relative quantitation of the 4 reporter ions for each protein. For protein identification and quantitative profiling, a minimum of 2 peptides at 99% or greater confidence was required. After confident protein profiles were collected for each of the 5 pairwise comparisons of the SRNS and the SSNS samples, the collective proteins from across all 5 groups were analyzed using the vendor supplied (Sciex) Protein Alignment Template algorithm (v.2.000p). This algorithm allows for the comparison of up to 10 pairwise groups to determine common protein changes across all the groups. The data reported here required that the proteins be detected in a minimum of 3 of the 5 samples and maintained statistically significance of p<0.05 based on a t-test versus the null values.

Urine Measurements

Urine Vitamin D Binding Protein was measured using a commercially available ELISA (R&D Systems, Minneapolis, Minn.). Intra and inter-assay CVs were 5.9% and 6.2%, respectively. The urine Neutrophil Galactase-Associated Lipocalin ELISA was performed using a commercially available assay (NGAL ELISA Kit 036; Bioporto, Grusbakken, Denmark) that specifically detects human NGAL. The intra-assay coefficient of variation (cv's) was 2.1% and inter-assay variation was 9.1%. Alpha 1 acid glycoprotein-2 (AGP2 or orosomucoid 2) was measured using a commercially available ELISA (Abnova—Taipei City, Taiwan) with an intra-assay CV of 4.4% and an inter-assay CV of 7.2%. Human Fetuin-A and Alpha-1 Acid Glycoprotein-1 (AGP, or orosomucoid) were measured using commercially available ELISAs with CVs (intra/inter) of 5.5%/7.6% and 5.6%/7.2%, respectively. Human thyroxine binding globulin (TBG) was measured using a commercially available ELISA (Kamiya Biomedical, Seattle, Wash.). TBG had CV's of 8.2% (intra) and 10.1% (inter). Hemopexin and Prealbumin (transthyretin) were measured with commercially available ELISAs (Assaypro, St. Charles, Mo.) with CV's of (intra/inter) of 4.9%/7.3% and 4.6%/9.0% respectively. Alpha-2 macroglobulin was measured using immunonephelometry on a Siemens BNII clinical nephelometer (Siemens, Munich, Germany) Alpha 1B glycoprotein was measured using a lab constructed ELISA as described previously. [13]

Statistical Analysis on Selected Biomarkers Measured at the Patient Level.

Ten biomarkers selected after Quantitative Protein Profiling were further measured at the patient level using a total of 50 patients, 20 with SRNS and 30 with SSNS. Since all the biomarkers showed right skewness of their empirical distributions, log −2 transformations were used to correct the skewness and ensure that parametric statistical models could be used in analyses. Means with original values were presented after taking inverse function of the transformed means (i.e. 2 transformed mean) estimated from the statistical models. Two steps of statistical analyses were used in the study. In Step 1 of analysis of association, each biomarker was compared of its means between SRNS and SSNS groups using two sample tests. In Step 2 of predictive analysis, multivariate logistical regression models were used to predict SRNS using a panel of biomarkers. Here, Applicant considered two candidate panels, one that employed all 10 biomarkers as the panel (or the predictors) in the logistical model (MLM-10), and the other that chose 5 biomarkers that showed significance in Step 1 (MLM-5). The multivariate logistical regression model from each panel would calculate a logit or risk score of SRNS and the score was evaluated for discriminative or diagnostic accuracy of SRNS using a ROC curve. In particular, the overall accuracy could be evaluated using the area under the ROC curve (or AUC) and specific accuracy under a cut off score could be evaluated using corresponding sensitivity and specificity. The accuracy is considered “outstanding”, “excellent”, “very good”, “fair” and “poor” if an AUC is “0.9-1”, “0.8-0.89”, “0.7-0.79”, “0.6-0.69”, and “<0.6” respectively, and a sensitivity or specificity is “0.8-1”, “0.6-0.79”, “0.4-0.59”, “0.2-0.39”, and “<0.2” respectively. The comparison between a ROC curve from a multivariate model vs. a ROC curve from an individual biomarker was tested using a non-parametric test. [14] The same analyses were repeated in a sub set of relapsed patients only. Sub analyses on relapsed patients were not performed given too small the sample size, especially those with SRNS (N=3). All statistical analyses were performed using SAS 9.4 software (SAS, Cary, N.C.). P-values <0.05 were considered statistically significant.

Results

Patients

Fifty patients were enrolled over a 24-month period. Of those 50 patients, 20 had SRNS, 16 of which had biopsy proven FSGS. 17 patients had active disease and 3 were in remission. 30 patients responded to steroid treatment and were labeled SSNS at the time of urine collection, 14 SSNS patients were in relapse, and 16 were in remission. 17 SRNS patients and the active SSNS patients had 4+ proteinuria readings by dipstick at time of collection. The 4+ reading is indicative of a protein concentration greater than 2000 mg/dl. Table 1 displays the patient demographics. SRNS differed from SSNS in terms of age (12.3 vs 7.5 years, p<0.001), pathology (FSGS vs no biopsy, respectively, p<0.001), presence of hypertension (75% vs 30%, respectively, p=0.003) and steroid treatment (SRNS 45% vs SSNS 87%, p=0.001).

TABLE 1
Patient Demographics

| | SRNS | | SSNS | | P- |
|---|---|---|---|---|---|
| Variable | (n = 20) | | (n = 30) | | value |
| Age (years; mean ± SE) | 12.3 ± 1.2 | | 7.5 ± 0.8 | | 0.001 |
| Sex (%) Male | 14 | (70) | 20 | (68) | NS |
| Pathology (%) | | | | | |
| FSGS | 16 | (80) | 2 | (6.7) | 0.001 |
| MCD | 1 | (5) | 7 | (23.3) | |
| Other | 2 | (10) | 0 | | |
| No biopsy | 1 | (5) | 21 | (70) | |
| Hypertension (%) | 15 | (75) | 9 | (30) | 0.003 |
| Immunosuppressant (%) | | | | | |
| Steroid | 9 | (45) | 26 | (87) | 0.001 |
| CNI | 4 | (20) | 5 | (17) | |
| MMF | 3 | (15) | 1 | (3) | |
| Rituximab | 2 | (10) | 4 | (13) | |
| CTX | 2 | (10) | 3 | (10) | |
| ACEI/ARB (%) | 8 | (40) | 1 | (3) | NA |

| | | | |
|---|---|---|---|
| GFR (ml/min/1.73 m2) | 119 ± 11.4 | 135 ± 6.1 | NS |
| MALB/Cr (mg/mg; ±SE)* | 2.0 ± 0.6 | 1.5 ± 0.34 | NS |

iTRAQ Profiling for Differential Proteins in SRNS Versus SSNS.

Samples from a cohort of ten patients (5 in each group) were prepared in duplicate (see Table 2) using a 4-plex isotope tagging method (iTRAQ) followed by nanoLC-MSMS profiling of the sample groups for protein identification and evaluation of quantitative changes as described in the experimental section. Collectively over 150 proteins were identified from the sample sets. Of these 150+ proteins identified, 72 proteins were identified and quantified in at least 3 of the 5 pairwise groups. Importantly, statistical analysis the protein changes among the patient cohort revealed 13 protein changes with p values <0.05. (Table 3). These 13 proteins were selected for further validation in a larger sample group.

TABLE 2

| 4-plex Group | Sample ID | Tag | Sample Group |
|---|---|---|---|
| A | SRNS, Sample 007, rep 1 | 114 | Resistant |
| | SRNS, Sample 007, rep 2 | 115 | |
| | SSNS, Sample 002, rep 1 | 116 | Sensitive |
| | SSNS, Sample 002, rep 2 | 117 | |
| B | SRNS, Sample 008, rep 1 | 114 | Resistant |
| | SRNS, Sample 008, rep 2 | 115 | |
| | SSNS, Sample 015, rep 1 | 116 | Sensitive |
| | SSNS, Sample 015, rep 2 | 117 | |
| C | SRNS, Sample 009, rep 1 | 114 | Resistant |
| | SRNS, Sample 009, rep 2 | 115 | |
| | SSNS, Sample 027, rep 1 | 116 | Sensitive |
| | SSNS, Sample 027, rep 2 | 117 | |
| D | SRNS, Sample 012, rep 1 | 114 | Resistant |
| | SRNS, Sample 012, rep 2 | 115 | |
| | SSNS, Sample 021, rep 1 | 116 | Sensitive |
| | SSNS, Sample 021, rep 2 | 117 | |
| E | SRNS, Sample 004, rep 1 | 114 | Resistant |
| | SRNS, Sample 004, rep 2 | 115 | |
| | SSNS, Sample 013, rep 1 | 116 | Sensitive |
| | SSNS, Sample 013, rep 2 | 117 | |

TABLE 3

| | | SSNS/ | | | | | | |
|---|---|---|---|---|---|---|---|---|
| | | SRNS | SSNS/SRNS | SSNS/SRNS | SSNS/SRNS | SSNS/SRNS | Average | |
| Accession | Protein Name | Group A | Group B | Group C | Group D | Group E | Log2 | p-value |
| sp\|P02774\| | Vitamin D- | −0.668 | −0.529 | −0.628 | −0.078 | −0.919 | −0.564 | 0.015 |
| VTDB_HUMAN | binding protein | | | | | | | |
| | (VDBP) | | | | | | | |
| sp\|P02765\|FETUA_HUMAN | Fetuin A | −0.530 | −0.466 | −0.365 | | −0.278 | −0.410 | 0.005 |
| sp\|P02790\|HEMO_HUMAN | Hemopexin | −0.328 | −0.366 | −0.337 | | −0.554 | −0.396 | 0.005 |
| sp\|P02766\|TTHY_HUMAN | Prealbumin | −0.281 | −0.531 | −0.326 | −0.052 | −0.491 | −0.336 | 0.017 |
| sp\|P02647\|APOA1_HUMAN | Apolipoprotein | −0.139 | −0.368 | −0.332 | −0.186 | −0.575 | −0.320 | 0.014 |
| | A-1 | | | | | | | |
| sp\|P01019\|ANGT_HUMAN | Angiotensinogen | −0.263 | −0.225 | −0.260 | | −0.376 | −0.281 | 0.003 |
| sp\|P01024\|CO3_HUMAN | Complement | −0.323 | −0.098 | −0.208 | −0.059 | −0.211 | −0.180 | 0.018 |
| | C3 | | | | | | | |
| sp\|P01023\|A2MG_HUMAN | Alpha-2 | −0.139 | −0.175 | | −0.172 | | −0.162 | 0.005 |
| | macroglobulin | | | | | | | |
| sp\|P02763\|A1AG1_HUMAN | Alpha-1 acid | 0.140 | 0.177 | 0.183 | 0.101 | 0.086 | 0.138 | 0.002 |
| | glycoprotein 1 | | | | | | | |
| | (AGP1) | | | | | | | |
| sp\|P05543\|THGB_HUMAN | Thyroxine- | 0.126 | 0.240 | 0.349 | 0.213 | 0.056 | 0.197 | 0.017 |
| | binding | | | | | | | |
| | globulin (TBG) | | | | | | | |
| sp\|P19652\|A1AG2_HUMAN | Alpha-1 acid | 0.238 | 0.066 | 0.317 | 0.459 | 0.247 | 0.265 | 0.014 |
| | glycoprotein 2 | | | | | | | |
| | (AGP2) | | | | | | | |
| sp\|P25311\|ZA2G_HUMAN | Zinc-alpha-2 | 0.205 | −0.013 | 0.529 | 0.437 | 0.366 | 0.305 | 0.033 |
| | glycoprotein | | | | | | | |
| sp\|P04217\|A1BG_HUMAN | Alpha-1B | 0.120 | 0.324 | 0.733 | 0.681 | 0.173 | 0.406 | 0.033 |
| | glycoprotein | | | | | | | |

Validation

Of the 13 proteins determined to be different between the 2 groups, Applicant found reliable assays for 9 proteins. These proteins were included for validation using ELISA or immunonephelometry in the expanded cohort (n=50): AGP, AGP2, Alpha-1 microglobulin, A1BG, Fetuin-A, Hemopexin, Prealbumin (transthyretin), TBG, VDBP. In addition, we measured NGAL because we have previously shown it to be able to differentiate SSNS from SRNS. [15] Table 4 shows that VDBP (p<0.001), prealbumin (p<0.001), NGAL (p=0.001), fetuin A (p<0.001) and AGP2 (p=0.03) were all 5.5-38-fold higher in SRNS patients than SSNS in the complete cohort.

TABLE 4
Summary of biomarkers by SSNS/SRNS

| | | | | | Fold | |
|---|---|---|---|---|---|---|
| | Mean (95% CI) | | | | (SRNS/ | |
| Var | SRNS | | SSNS | | SSNS) | p |
| All (N = 50) | | | | | | |
| | N = 20 | | N = 30 | | | |
| VDBP | 2,519.41 | (669.59, 9,479.56) | 66.25 | (22.46, 195.47) | 38.0 | <0.001 |
| NGAL | 30.77 | (15.01, 63.08) | 5.57 | (3.10, 10.00) | 5.5 | 0.001 |
| Fetuin A | 36,723.78 | (13,878.94, 97,171.38) | 3,433.82 | (1,551.44, 7,600.15) | 10.7 | <.001 |
| Prealbumin | 20,685.39 | (7,391.11, 57,891.95) | 1,649.83 | (712.04, 3,822.76) | 12.5 | <.001 |
| AGP2 | 141.30 | (54.38, 367.14) | 35.79 | (16.41, 78.04) | 3.9 | 0.030 |
| AGP1 | 90.97 | (13.43, 616.16) | 82.89 | (17.38, 395.22) | 1.1 | 0.940 |
| A2MCG | 119.93 | (40.33, 356.62) | 35.79 | (14.70, 87.13) | 3.4 | 0.090 |
| A1BG | 310.97 | (146.86, 658.43) | 192.57 | (104.37, 355.31) | 1.6 | 0.325 |
| TBG | 1,136.19 | (320.34, 4,029.90) | 730.91 | (259.97, 2,054.98) | 1.6 | 0.590 |
| Hemopexin | 4,701.67 | (1,993.48, 11,089.00) | 2,049.40 | (1,017.11, 4,129.39) | 2.3 | 0.138 |
| Relapse (N = 31) | | | | | | |
| | N = 17 | | N = 14 | | | |
| VDBP | 3,708.40 | (1,010.16, 13,613.90) | 353.58 | (84.36, 1,482.06) | 10.5 | 0.018 |
| NGAL | 33.48 | (15.22, 73.64) | 7.16 | (3.00, 17.06) | 4.7 | 0.011 |
| Fetuin A | 55,745.38 | (23,435.74, 132,598.64) | 15,607.72 | (6,006.81, 40,554.13) | 3.6 | 0.053 |
| Prealbumin | 33,079.70 | (12,129.94, 90,212.00) | 5,000.48 | (1,655.35, 15,105.43) | 6.6 | 0.014 |
| AGP2 | 171.01 | (81.37, 359.43) | 266.72 | (117.65, 604.69) | 0.6 | 0.422 |
| AGP1 | 141.97 | (22.88, 881.03) | 1,340.72 | (179.35, 10,022.32) | 0.1 | 0.103 |
| A2MCG | 137.11 | (44.26, 424.79) | 110.19 | (31.70, 383.10) | 1.2 | 0.795 |
| A1BG | 318.05 | (139.00, 727.74) | 241.52 | (97.01, 601.29) | 1.3 | 0.655 |
| TBG | 1,639.78 | (419.97, 6,402.53) | 1,237.83 | (275.92, 5,553.08) | 1.3 | 0.781 |
| Hemopexin | 4,019.45 | (1,583.99, 10,199.55) | 3,126.86 | (1,120.64, 8,724.72) | 1.3 | 0.717 |

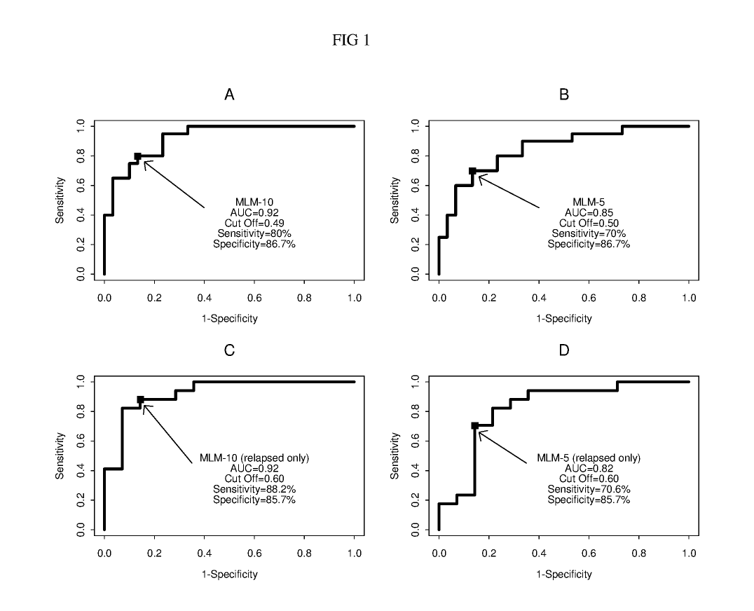

The predictive analyses showed the panel of biomarkers (MLM-5) improved the AUC to 0.85, significantly higher than that of AGP2 or any individual biomarker not selected in the panel. The panel using all 10 biomarkers (MLM-10) yielded an AUC of 0.92, significantly higher than that of any single biomarker (Table 5). Sensitivities and specificities from panels showed excellent—outstanding accuracy under suggested cut off scores (Table 6 and FIG. 1). Table 7 provides the algorithms to calculate the risk scores of SRNS of the panels. Similar conclusions could be reached in the sub analyses on relapsed patients.

TABLE 5
Summary of AUC, sensitivity and specificity of detecting SSNS

| ROCModel | AUC (95% CI) | p vs. MVM_10 | p vs. MVM_5 |
|---|---|---|---|
| All (N = 50) | | | |
| MLM-10 | 0.92 (0.85, 0.99) | — | 0.076 |
| MLM-5 | 0.85 (0.74, 0.96) | 0.076 | — |
| VDBP | 0.81 (0.68, 0.95) | 0.052 | 0.267 |
| NGAL | 0.78 (0.65, 0.91) | 0.020 | 0.264 |
| Fetuin A | 0.78 (0.65, 0.91) | 0.016 | 0.195 |
| Prealbumin | 0.78 (0.65, 0.91) | 0.026 | 0.286 |
| AGP2 | 0.65 (0.49, 0.80) | 0.001 | 0.011 |
| AGP1 | 0.55 (0.39, 0.71) | 0.000 | 0.000 |
| A2MCG | 0.64 (0.48, 0.80) | 0.001 | 0.027 |
| A1BG | 0.59 (0.42, 0.75) | 0.000 | 0.008 |
| TBG | 0.56 (0.39, 0.73) | 0.000 | 0.003 |
| Hemopexin | 0.66 (0.50, 0.82) | 0.002 | 0.028 |
| Relapse (N = 31) | | | |
| MLM-10 | 0.92 (0.83, 1.00) | — | 0.129 |
| MLM-5 | 0.82 (0.66, 0.99) | 0.129 | — |
| VDBP | 0.77 (0.58, 0.96) | 0.105 | 0.561 |
| NGAL | 0.76 (0.58, 0.94) | 0.037 | 0.312 |
| Fetuin A | 0.68 (0.48, 0.88) | 0.016 | 0.118 |
| Prealbumin | 0.73 (0.55, 0.91) | 0.035 | 0.215 |
| AGP2 | 0.60 (0.39, 0.80) | 0.003 | 0.067 |
| AGP1 | 0.57 (0.35, 0.79) | 0.002 | 0.091 |
| A2MCG | 0.52 (0.30, 0.73) | 0.001 | 0.023 |
| A1BG | 0.58 (0.36, 0.79) | 0.005 | 0.051 |
| TBG | 0.57 (0.36, 0.78) | 0.003 | 0.045 |
| Hemopexin | 0.56 (0.35, 0.77) | 0.003 | 0.080 |

TABLE 6
Sensitivity and specificity of detecting SSNS using
suggest cut offs from multivariate logistic models

| Model | AUC | Cut off prob | Sens | SPEC |
|---|---|---|---|---|
| All (N = 50) | | | | |
| MLM-10 | 0.92 (0.85, 0.99) | 0.49 | 80.0% | 86.7% |
| MLM-5 | 0.85 (0.74, 0.96) | 0.50 | 70.0% | 86.7% |
| Relapse (N = 31) | | | | |
| MLM-10 | 0.92 (0.83, 1.00) | 0.60 | 88.2% | 85.7% |
| MLM-5 | 0.82 (0.66, 0.99) | 0.60 | 70.6% | 85.7% |

TABLE 7
Algorithms of computing risk scores of SRNS

| | | | COLUMN 3 | COLUMN 4 |
|---|---|---|---|---|
| | COLUMN 1 | COLUMN 2 | MLM-10 (relapsed | MLM-5 (relapsed |
| Step | MLM-10 (any patient) | MLM-5 (any patient) | patient only) | patient only) |
| 1 | converting all | converting all | converting all | converting all |
| | biomarkers into | biomarkers into | biomarkers into | biomarkers into |
| | log2 values | log2 values | log2 values | log2 values |
| 2 | Each biomarker is | Each biomarker is | Each biomarker is | Each biomarker is |
| | adjusted by a | adjusted by a | adjusted by a | adjusted by a |
| | multiplier in the | multiplier in the | multiplier in the | multiplier in the |
| | following: | following: | following: | following: |
| | 0.27 × VDBP | 0.23 × VDBP | 0.23 × VDBP | 0.14 × VDBP |
| | −0.004 × Prealbumin | −0.03 × Prealbumin | 0.07 × Prealbumin | 0.16 × Prealbumin |
| | 0.51 × NGAL | 0.32 × NGAL | 0.82 × NGAL | 0.26 × NGAL |
| | 0.37 × Fetuin A | 0.01 × Fetuin A | 0.41 × Fetuin A | −0.05 × Fetuin A |
| | 0.50 × AGP2 | 0.03 × AGP2 | 0.42 × AGP2 | −0.47 × AGP2 |
| | −0.49 × AGP1 | | −0.60 × AGP1 | |
| | 0.22 × A2MCG | | 0.24 × A2MCG | |
| | 0.20 × Hemopexin | | 0.36 × Hemopexin | |
| | −0.09 × TBG | | −0.15 × TBG | |
| | 0.11 × A1BG | | 0.24 × A1BG | |
| 3 | Sum the adjusted | Sum the adjusted | Sum the adjusted | Sum the adjusted |
| | biomarkers | biomarkers | biomarkers | biomarkers |
| 4 | Calculate a raw | Calculate a raw | Calculate a raw | Calculate a raw |
| | score by subtracting | score by subtracting | score by subtracting | score by subtracting |
| | 13.65 from the sum in | 3.58 from the sum in | 17.27 from the sum in | 0.03 from the sum in |
| | Step 3. | Step 3. | Step 3. | Step 3. |
| 5 | Calculate the risk | Calculate the risk | Calculate the risk | Calculate the risk |
| | score by taking 2 to | score by taking 2 to | score by taking 2 to | score by taking 2 to |
| | the power of the raw | the power of the raw | the power of the raw | the power of the raw |
| | score in Step 4. | score in Step 4. | score in Step 4. | score in Step 4. |
| 6 | Compare the risk | Compare the risk | Compare the risk | Compare the risk |
| | score to the cutoff | score to the cutoff | score to the cutoff | score to the cutoff |
| | point 0.49: | point 0.50: | point 0.60: | point 0.60: |
| | SRNS positive if the | SRNS positive if the | SRNS positive if the | SRNS positive if the |
| | score > cut off; | score > cut off; | score > cut off; | score > cut off; |
| | SRNS negative if the | SRNS negative if the | SRNS negative if the | SRNS negative if the |
| | score ≤ cut off. | score ≤ cut off. | score ≤ cut off. | score ≤ cut off. |

Discussion

Steroid resistant nephrotic syndrome (SRNS) is significantly associated with poor outcome when compared to steroid sensitive nephrotic syndrome (SSNS). [16-19] The incidence of SRNS is on the rise, as marked by the increase in incidence of FSGS in children. [20-22] Currently, the only method of diagnosis is an invasive biopsy which is not typically performed in children until first line treatments fail. This results in patients with SRNS getting an unnecessary exposure to high dose corticosteroids and a delay in initiating a more appropriate treatment. In this study, our objective was to use a robust proteomic technique, iTRAQ, to identify potential biomarkers that could be used to non-invasively distinguish steroid resistant patients from those whose disease is likely to respond to steroids. Of the thirteen differentially expressed proteins identified by iTRAQ, we were able to use ELISA and immunonephelometry to validate a 10-biomarker panel with a high discriminatory power to identify SRNS (AUC 0.92) in both the complete cohort and the subset with active disease. In addition, we demonstrated that by using the 5 markers with significant association to SRNS, we were still able to achieve an AUC of 0.85 in the complete cohort, and an AUC of 0.82 in the active disease subjects. This predictive biomarker panel includes VDBP, NGAL, fetuin A, prealbumin, and AGP2.

The current study adds further validation to the previous findings concerning VDBP and NGAL in SRNS. VDBP and NGAL have previously been shown to be increased in children with SRNS.[15, 23] While both had been shown previously to be correlated with proteinuria [24, 25], their ability to distinguish SRNS from SSNS was independent of proteinuria as measured by MALB/Cr.[23] It was found that VDBP by itself showed high discriminatory power (AUC 0.87, p<0.0002) between SRNS and SSNS patients, independent of proteinuria, indicating that there may be a disease specific process leading to increased uVDBP (urinary VDBP) in SRNS patients. One plausible explanation relates to the fact that reabsorption of any filtered VDBP requires the integrity of megalin and cubulin receptors in the proximal tubule. Thus, any form of chronic tubular injury, as would be expected in SRNS, could result in increased uVDBP excretion. Supporting this theory, VDBP was recently shown to be a potential marker of tubular fibrosis and renal interstitial damage in a rat model of adriamycin-induced nephrosis. [26]

Interestingly, fetuin-A has been shown to work through megalin mediated endocytosis to counter nephrocalcification in the tubular lumen in rats. [27] Therefore, like VDBP, increased excretion of fetuin-A in the urine could be explained by megalin disfunction, and could represent a mechanism for the appearance of these proteins at high levels in the urine of SRNS patients. It has been suggested by some that the normal glomerulus leaks proteins at a nephrotic level, but that those proteins are generally reabsorbed in the proximal tubule by megalin and related proteins. [28] However, in the nephrotic kidney, levels of megalin, clathrin and other important parts of the endocytic pathway are compromised, which leads to albuminuria. Given the number of podocyte mutations discovered in nephrotic diseases such as FSGS, [29] it is unlikely that megalin disfunction accounts for all aspects of the disease, but it remains an intriguing possibility given some of our findings. Fetuin A has been demonstrated to be a potential marker for other renal conditions as well. Inoue, et al. [30], demonstrated that fetuin-A was a risk factor for both microalbuminuria and reduction of GFR in diabetic nephropathy, and could therefore be utilized as a marker to predict progression of the disease. Urinary fetuin-A has also been shown to be a sensitive (94%), yet not especially specific (60%) marker for progression and prediction of renal insufficiency in autosomal dominant polycystic kidney disease. Fetuin A, similar to NGAL, appears to be a sensitive marker of progression of disease, but lacks some specificity to individual disease processes. [31, 32]

Not all the markers we discovered have a track record as being associated with CKD in the urine. For instance, serum prealbumin levels are often elevated in chronic kidney disease and are used to evaluate nutritional status in dialysis patients [33], but urinary levels have not appeared associated with specific diseases in the literature.

Applicant notes that this was a single center, cross-sectional pilot study with a small group of patients who had already begun treatment at enrollment. This limits the conclusions we can draw about the value of our biomarker panel to predict steroid responsiveness in NS patients. There is also a significant age difference between our SRNS patients and our SSNS patients. This is inherent in any study of nephrotic syndrome since the majority of patients in the SSNS have MCD and the majority of SRNS patients have FSGS. Approximately 70% of children with MCD are under 5 years of age, while primary FSGS is typically not diagnosed until after the age of 6. [2] However, with such a high discriminatory power (AUC 0.93, p<0.0001), our results are unlikely to represent an artifact of age differences. Since serum samples were not available to our research team, we were unable to verify that any of the markers were elevated due to elevation in the blood and leaking into the urine. Within the clinical context of a child with NS, our results indicate promising utility of this biomarker panel for the discrimination of the steroid resistant form of the disease.

The disclosed biomarker panel may for the prediction of response to treatment and obviate the need for unnecessary exposure to high dose corticosteroids and other powerful immunosuppressants in patients who are unlikely to respond. Biomarkers may be used as surrogate endpoints, and are valuable in clinical trials and can allow for more rapid drug development. The discovery of a urinary panel that could predict response to treatment in nephrotic syndrome could aid the physician in developing an individualized treatment plan that could potentially lead to better care for patients with this serious and progressive disease.

### REFERENCES

1. Gipson, D. S., et al., Management of Childhood Onset Nephrotic Syndrome. Pediatrics, 2009. 124(2): p. 747-757.
2. Eddy, A. A. and J. M. Symons, Nephrotic syndrome in childhood. Lancet, 2003. 362(9384): p. 629-39.
3. Smith, J. M., et al., Contributions of the Transplant Registry: The 2006 Annual Report of the North American Pediatric Renal Trials and Collaborative Studies (NAPRTCS). Pediatr Transplant, 2007. 11(4): p. 366-73.
4. Korbet, S. M., Clinical picture and outcome of primary focal segmental glomerulosclerosis. Nephrol Dial Transplant, 1999. 14(suppl 3): p. 68-73.
5. Gulati, S., et al., Do current recommendations for kidney biopsy in nephrotic syndrome need modifications? Pediatr Nephrol, 2002. 17(6): p. 404-8.
6. Neuhaus, T. J., et al., Alternative treatment to corticosteroids in steroid sensitive idiopathic nephrotic syndrome. Arch Dis Child, 1994. 71(6): p. 522-6.
7. Gygi, S. P., et al., Quantitative analysis of complex protein mixtures using isotope-coded affinity tags. Nat Biotechnol, 1999. 17(10): p. 994-9.
8. Ross, P. L., et al., Multiplexed protein quantitation in Saccharomyces cerevisiae using amine-reactive isobaric tagging reagents. Mol Cell Proteomics, 2004. 3(12): p. 1154-69.
9. Schwartz, G. J., et al., New equations to estimate GFR in children with CKD. J Am Soc Nephrol, 2009. 20(3): p. 629-37.
10. National Kidney, F., K/DOQI clinical practice guidelines for chronic kidney disease: evaluation, classification, and stratification. Am J Kidney Dis, 2002. 39(2 Suppl 1): p. S1-266.
11. Eismann, T., et al., Peroxiredoxin-6 protects against mitochondrial dysfunction and liver injury during ischemia-reperfusion in mice. Am J Physiol Gastrointest Liver Physiol, 2009. 296(2): p. G266-74.
13. Piyaphanee, N., et al., Discovery and initial validation of alpha 1-B glycoprotein fragmentation as a differential urinary biomarker in pediatric steroid-resistant nephrotic syndrome. Proteomics Clin Appl, 2011. 5(5-6): p. 334-42.
14. DeLong, E. R., D. M. DeLong, and D. L. Clarke-Pearson, Comparing the areas under two or more correlated receiver operating characteristic curves: a nonparametric approach. Biometrics, 1988. 44(3): p. 837-45.
15. Bennett, M. R., et al., NGAL distinguishes steroid sensitivity in idiopathic nephrotic syndrome. Pediatr Nephrol, 2012. 27(5): p. 807-12.
16. Cattran, D. C. and P. Rao, Long-term outcome in children and adults with classic focal segmental glomerulosclerosis. Am J Kidney Dis, 1998. 32(1): p. 72-9.
17. Hari, P., et al., Treatment of focal glomerulosclerosis with pulse steroids and oral cyclophosphamide. Pediatr Nephrol, 2001. 16(11): p. 901-5.
18. Gipson, D. S., et al., Differential risk of remission and ESRD in childhood FSGS. Pediatr Nephrol, 2006. 21(3): p. 344-9.
19. Roberti, I. and S. Vyas, Long-term outcome of children with steroid-resistant nephrotic syndrome treated with tacrolimus. Pediatr Nephrol, 2010. 25(6): p. 1117-24.
20. Gulati, S., et al., Changing trends of histopathology in childhood nephrotic syndrome. Am J Kidney Dis, 1999. 34(4): p. 646-50.
21. Bonilla-Felix, M., et al., Changing patterns in the histopathology of idiopathic nephrotic syndrome in children. Kidney Int, 1999. 55(5): p. 1885-90.
22. Srivastava, T., S. D. Simon, and U. S. Alon, High incidence of focal segmental glomerulosclerosis in nephrotic syndrome of childhood. Pediatr Nephrol, 1999. 13(1): p. 13-18.
23. Bennett, M. R., et al., Urinary Vitamin D-Binding Protein as a Biomarker of Steroid-Resistant Nephrotic Syndrome. Biomark Insights, 2016. 11: p. 1-6.
24. Grymonprez, A., et al., Vitamin D metabolites in childhood nephrotic syndrome. Pediatr Nephrol, 1995. 9(3): p. 278-81.
25. Doorenbos, C. R., et al., Antiproteinuric treatment reduces urinary loss of vitamin D-binding protein but does not affect vitamin D status in patients with chronic kidney disease. J Steroid Biochem Mol Biol, 2012. 128(1-2): p. 56-61.
26. Mirkovic, K., et al., Urinary vitamin D binding protein: a potential novel marker of renal interstitial inflammation and fibrosis. PLoS One, 2013. 8(2): p. e55887.
27. Matsui, I., et al., Retention of fetuin-A in renal tubular lumen protects the kidney from nephrocalcinosis in rats. Am J Physiol Renal Physiol, 2013. 304(6): p. F751-60.
28. Russo, L. M., et al., The normal kidney filters nephrotic levels of albumin retrieved by proximal tubule cells: retrieval is disrupted in nephrotic states. Kidney Int, 2007. 71(6): p. 504-13.
29. Lovric, S., et al., Genetic testing in steroid-resistant nephrotic syndrome: when and how? Nephrol Dial Transplant, 2015.
30. Inoue, K., et al., Urinary fetuin-A is a novel marker for diabetic nephropathy in type 2 diabetes identified by lectin microarray. PLoS One, 2013. 8(10): p. e77118.
31. Wu, J., et al., Urinary TNF-alpha and NGAL are correlated with the progression of nephropathy in patients with type 2 diabetes. Exp Ther Med, 2013. 6(6): p. 1482-1488.
32. Bolignano, D., et al., Neutrophil gelatinase-associated lipocalin (NGAL) and progression of chronic kidney disease. Clin J Am Soc Nephrol, 2009. 4(2): p. 337-44.
33. Rymarz, A., et al., The Associations Between Body Cell Mass and Nutritional and Inflammatory Markers in Patients With Chronic Kidney Disease and in Subjects Without Kidney Disease. J Ren Nutr, 2015.

All percentages and ratios are calculated by weight unless otherwise indicated.

All percentages and ratios are calculated based on the total composition unless otherwise indicated.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “20 mm” is intended to mean “about 20 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method for identifying an individual likely to have steroid resistant nephrotic syndrome (SRNS), comprising the step of detecting a plurality of proteins comprising Vitamin D Binding Protein (VDBP), Alpha-1 Acid Glycoprotein 2 (AGP-2), Fetuin A, prealbumin, and NGAL in a urine sample obtained from said individual.

2. The method of claim 1, further comprising the step of quantifying said plurality of proteins.

3. The method of claim 1, further comprising the step of quantifying said plurality of proteins and identifying the presence of an alteration in the level of each of said plurality of proteins, wherein an alteration is indicative of said individual being steroid sensitive or steroid resistant.

4. The method of claim 1, further comprising the step of quantifying said plurality of proteins and applying the algorithm set forth in Column 2 of Table 7.

5. The method of claim 1, wherein said detection step is carried out using a 4-plex isotope tagging method (iTRAQ)

6. The method of claim 1, further comprising detecting Alpha-1 Acid Glycoprotein 1 (AGP-1), Alpha-1 B Glycoprotein (A1BG), Thyroxine binding globulin, hemopexin, and alpha-2 macroglobulin in said sample.

7. The method of claim 1, further comprising the step of quantifying said proteins

8. The method of claim 1, further comprising the step of quantifying said proteins, and applying the algorithm set forth in Column 1 of Table 7.

9. The method of claim 1, further comprising the step of quantifying said proteins, and applying the algorithm set forth in Column 1 of Table 7, wherein said individual is characterized as either steroid resistant or steroid sensitive.

10. The method of claim 1, further comprising the step of quantifying said proteins, and applying the algorithm set forth in Column 1 of Table 7, wherein said individual is characterized as either steroid resistant or steroid sensitive, wherein said algorithm is computer-implemented.

11. The method of claim 1, wherein said individual is diagnosed with nephrotic syndrome.

12. The method of claim 1, wherein said detection step is carried out using a 4-plex isotope tagging method (iTRAQ)

13. A method of identify a steroid resistant individual diagnosed with nephrotic syndrome, comprising the step of contacting a urine sample from said individual with a composition comprising a plurality of detecting agents, wherein said plurality of detecting agents are capable of detecting a protein selected from Vitamin D Binding Protein (VDBP), Alpha-1 Acid Glycoprotein 2 (AGP-2), Fetuin A, prealbumin, and NGAL.

14. The method of claim 13, wherein said composition further comprises a plurality of detecting agents capable of detecting a protein selected from Alpha-1 Acid Glycoprotein 1 (AGP-1), Alpha-1 B Glycoprotein (A1BG), Thyroxine binding globulin, hemopexin, and alpha-2 macroglobulin.

15. The method of claim 13, wherein said detecting agent is an antibody.

16. The method of claim 14, wherein said detecting agent is an antibody.

17. A kit for classifying a subject diagnosed with nephrotic syndrome as steroid sensitive or steroid sensitive, comprising a set of detection agents consisting of detection agents capable of detecting the expression products of 5 different biomarkers in a test sample, or 10 different biomarkers in a test sample, wherein said 5 different biomarkers are Vitamin D Binding Protein (VDBP), Alpha-1 Acid Glycoprotein 2 (AGP-2), Fetuin A, prealbumin, and NGAL and wherein said 10 different biomarkers are Vitamin D Binding Protein (VDBP), Alpha-1 Acid Glycoprotein 2 (AGP-2), Fetuin A, prealbumin, NGAL, Alpha-1 Acid Glycoprotein 1 (AGP-1), Alpha-1 B Glycoprotein (A1BG), Thyroxine binding globulin, hemopexin, and alpha-2 macroglobulin.

18. The kit of claim 17, further comprising a computer product for calculating a value for a subject according to Table 7, wherein said value is predictive of said classification.
